(19) United States
(12) Patent Application Publication
(10) Pub. No.: US 2009/0043244 A1
(43) Pub. Date: Feb. 12, 2009

(54) Electrotransport Drug Delivery Device Adaptable to Skin Resistance Change

(76) Inventor: Omer T. Inan, Stanford, CA (US)

Correspondence Address: Diehl Servilla LLC, 77 Brant Avenue, Suite 210, Clark, NJ, 07066, US

(21) Appl. No.: 12/185,852

(22) Filed: Aug. 5, 2008

Related U.S. Application Data
(60) Provisional application No. 60/954,766, filed on Aug. 8, 2007.

(51) Int. Cl. A61N 1/30; G05F 1/00

(52) U.S. Cl. 604/20; 323/265

(57) ABSTRACT

Disclosed is a transdermal electrotransport drug delivery system having a constant current that can accommodate large resistance change in a body surface. A semiconductor circuit component such as a Zener diode or a PMOS FET is used to impose a voltage drop from the output of a voltage booster circuit to maintain a constant current for electrotransport. Methods for its use are also disclosed.

### CROSS-REFERENCE TO RELATED APPLICATIONS

This application claims the benefit of U.S. Provisional Application No. 60/954,766, filed on Aug. 8, 2007, the content of which is hereby incorporated by reference in its entirety.

### TECHNICAL FIELD

The present invention relates to an electrotransport drug delivery system for delivering a drug across a body surface or membrane. In particular, the invention relates to a system that delivers a constant current over a period of time and adaptable to changes in skin resistance.

### BACKGROUND OF THE INVENTION

The delivery of active pharmaceutical agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

In transdermal drug delivery, the natural barrier function of the body surface, such as skin, mucosa, and the eye ball, presents a challenge to delivery therapeutics into circulation. Devices have been invented to provide transdermal delivery of drugs. Transdermal drug delivery can generally be considered to belong to one of two groups: transport by a “passive” mechanism or by an “active” transport mechanism. In the former, such as DUROGESIC® fentanyl transdermal systems (available from Jassen Pharmaceuticals) and other drug delivery skin patches, the drug is incorporated in a solid matrix, or a reservoir with rate-controlling membrane, and/or an adhesive system.

Passive transdermal drug delivery offers many advantages, such as ease of use, little or no pain at use, disposability, good control of drug delivery, and avoidance of hepatic first-pass metabolism. However, many active agents are not suitable for passive transdermal delivery because of their size, ionic charge characteristics, and hydrophilicity. Most passive transdermal delivery systems are not capable of delivering drugs under a specific profile, such as by ‘on-off’ mode, pulsatile mode, etc. Consequently, a number of alternatives have been proposed in which the flux of the drug(s) is driven by various forms of energy. Some examples include the use of iontophoresis, ultrasound, electroporation, heat and microneedles. These are considered to be “active” delivery systems.

One method for transdermal delivery of such active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art, see, e.g., U.S. Pat. Nos. 5,057,072; 5,084,008; 5,147,297; 5,373,242, 6,039,977; 6,049,733; 6,171,294, 6,181,963, 6,216,033; and U.S. Patent Publication No. 20030191946. In iontophoretic drug delivery, one electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A prior iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15. Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′,29 in adhesive 30. The skin-facing side 36 of the adhesive 30 has adequate adhesive property to maintain the device on the skin for the duration of the use of the device.

In iontophoresis, sometimes it is desirable that a constant electrical current is delivered to a pair of electrodes on the skin for a period of time to deliver the drug. The skin presents a dynamically varying electrical resistance, generally on the order of few to hundreds of kilo-Ohms (kΩ or kohm). Recently, there have been suggestions to boost the voltage to maintain a constant current through the load (i.e., the body tissue such as skin tissue through which the current passes to deliver the drug). Examples of iontophoretic delivery systems having booster circuits include U.S. Pat. Nos. 5,254,081, 5,804,957, and 6,842,640. However, we have found that there has not been any electrotransport system that is adaptable for the skin resistance falling to a small value. There have been suggestions of drug delivery with more sophisticated current or voltage profiles. See, for example, U.S. Pat. Nos. 5,207,752, 5,983,130, 6,219,576; WO 99/30773; and EP941085B1. However, there has not been an electrotransport system that has been shown to deliver drug with relatively stable current over time even when the load resistance (e.g., skin resistance in iontophoretic drug delivery) falls significantly. The present invention provides such a needed system.

### SUMMARY OF THE INVENTION

The present invention relates to an electrotransport device for delivering a drug through a body surface, such as the skin, of a patient with a constant current over a period of time. The present invention provides such electrotransport devices and methods of making and using such electrotransport devices. A semiconductor circuit component such as a Zener diode or a PMOS FET is used to impose a dynamic voltage drop from the output of a voltage booster circuit to maintain a constant current through the body surface. Hereinafter, skin will be used as the example of body surface.

In one aspect, the device has a donor reservoir including an electrotransportable drug, a first electrode and a second electrode for conducting a current to flow from the first electrode to the second electrode through the donor reservoir and the body surface (e.g., skin) to drive the electrotransportable drug from the donor reservoir transdermally, and a controller for delivering a constant current through the first and second electrodes. The controller contains a booster circuit that can boost the voltage of a power supply to a multiple of the voltage of the power supply to achieve a constant current. A feedback sensor and a semiconductor circuit component (preferably a discrete semiconductor circuit component) are connected electrically with the booster circuit and the first and second electrodes so that the same current flows through the feedback sensor, semiconductor circuit component and the electrodes. The feedback sensor provides a feedback voltage to the booster circuit for feedback control to result in a constant current during a period of time while accommodating changes in resistance through the skin. The semiconductor circuit component maintains the sum of voltage across the semiconductor circuit component, the skin, and the feedback sensor to be always at least equal to the voltage of the power supply.

In another aspect, a method for controlling current in a transdermal electrotransport device for delivery of a drug through the body surface (e.g., skin) is provided. The method includes boosting an input voltage with a booster circuit to a higher output voltage to achieve a constant current during a period of time while accommodating resistance changes of the body surface tissue to deliver a drug, wherein a semiconductor circuit component and a feedback sensor are connected electrically with the skin and the booster circuit so that the same current flows through the semiconductor circuit component, the feedback sensor and the body surface tissue. The feedback sensor is used for feedback control of the booster circuit to produce a constant current. The semiconductor circuit component imposes a dynamic voltage drop at constant current to maintain the sum of voltage across the semiconductor circuit component, the body surface tissue, and the feedback sensor to be always at least equal to the voltage of the power supply regardless of the resistance change in the body surface tissue.

We have found that in prior designs of electrotransport systems, with booster circuits boosting voltage to drive a constant current, if the body surface tissue resistance falls to a very low value, there is a risk of the system failing to maintain the current constant, which may result in delivering a larger current than desired. Through the use of a simple semiconductor circuit component it is possible to eliminate this risk. Semiconductor circuit components such as Zener diode or PMOS FET can be used for this purpose.

The semiconductor circuit component either imposes a constant voltage drop or an increasing voltage drop as the load resistance falls. Using a Zener diode in reverse bias at the output of a booster circuit imposes a constant voltage drop across the Zener diode regardless of the current or the voltage change at the output of the booster circuit. Thus, whether the load resistance changes or whether the constant level of the current is set or reset to different current levels, the voltage drop across the Zener diode will be the same, which leads to a very energy efficient system. Using a PMOS FET provides the advantage that the PMOS FET imposes an increasing voltage drop (due to increase in resistance of the PMOS FET) with a falling load resistance. Thus, during much of the operational range, the energy waste is low. The resistance of the PMOS FET and the energy dissipation thereof only go up an appreciable amount when the load resistance drops significantly. Thus, the system with PMOS FET is also very energy efficient. When the system has a means to set the level of the constant current to be delivered over different periods of time, the ability to impose a constant voltage or an increasing voltage resulting from a decrease in load resistance is advantageous. Regardless of what the setting of the current level is, the device will waste little energy in the range of normal operation. These semiconductor circuit components are superior in this application to a resistor or another passive component since the excess voltage drop is dynamically adjusted based on the need to maintain a constant current.

The present invention also provides methods of making and methods of using the above electrotransport devices.

The present invention is illustrated by way of examples in embodiments and not limitation in the figures of the accompanying drawn in which like references indicate similar elements. The figures are not shown to scale unless indicated otherwise in the content.

### BRIEF DESCRIPTION OF THE DRAWINGS

FIG. 1 illustrates an exploded perspective view of a prior art typical electrotransport system;

FIG. 2 is a block diagram representing an electrotransport system of the present invention;

FIG. 3 is a schematic representation of an embodiment of the present invention;

FIG. 4 illustrates a schematic representation of a boost converter that can be adapted to be used in an embodiment of the present invention;

FIG. 5 illustrates a schematic representation of a constant current system for delivery of a constant current to a load adaptable to be used in an embodiment of the present invention;

FIG. 6 illustrates a schematic representation of an embodiment of the present invention in which a Zener diode is used as the semiconductor circuit component in a current controller;

FIG. 7 illustrates a schematic representation of an embodiment of the present invention in which a Zener diode is used as the semiconductor circuit component in a current controller;

FIG. 8 shows a schematic representation of an embodiment of the present invention showing how the level of the constant current is adjusted; and

FIG. 9 illustrates a schematic representation of an embodiment of the present invention in which a PMOS FET is used as the semiconductor circuit component in a current controller.

### DETAILED DESCRIPTION OF THE INVENTION

The present invention is directed to an electrotransport drug delivery system that delivers drug with a constant current over a period of time. A constant current would tend to deliver a stable drug flux during that period. In particular, the system has a controller that controls the current delivery so that the device would not deliver a current larger than intended when the load resistance falls significantly.

The practice of the present invention will employ, unless otherwise indicated, conventional methods used by those skilled in the art of mechanical and electrical connections in drug device development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

As used herein, “dose period” refers to a period of time during which the device delivers a nominal dose that the device has been designed to deliver. Such a nominal dose is typically a target amount of drug that the device is specified to deliver according to regulatory approval by a competent government drug administration agency. Typically such a dose is delivered each time the device is activated for delivery of a dose.

As used herein, “semiconductor circuit component” is a device having semiconductor junction(s) such as p/n or n/p internally and the manner the device conducts electricity, such as the current, depends on the polarity of voltage applied across the junction. It is used as a component in a circuit and does not contain another circuit component, such as capacitor, inductor, resistor, transistor, and diode. A “discrete semiconductor circuit component” is a semiconductor component that is discrete, as opposite to being incorporated into an integrated circuit.

As used herein, “switch” when referring to a voltage booster circuit means a semiconductor circuit component (such as a transistor) that can open or close to allow or stop current flow therethrough. A “switch regulator” refers to a device that uses switch(s) to regulate voltage.

As used herein, “boost converter” means an electronic circuit having semiconductor switch and energy storage such as an inductor for stepping up an input voltage to achieve an output voltage higher than the input voltage. Generally a boost converter may also contain a rectification component (diode).

The present invention provides an electrotransport device that is for electrotransport delivery of a drug through a body surface, e.g., skin, such as a system that contains fentanyl salt (e.g., fentanyl HCl) or sufentanil salt (e.g., sufentanil HCl or sufentanil citrate). In a system of the present invention, a semiconductor circuit component prevents the output voltage of a booster circuit to ever fall below that of the input voltage of the booster circuit.

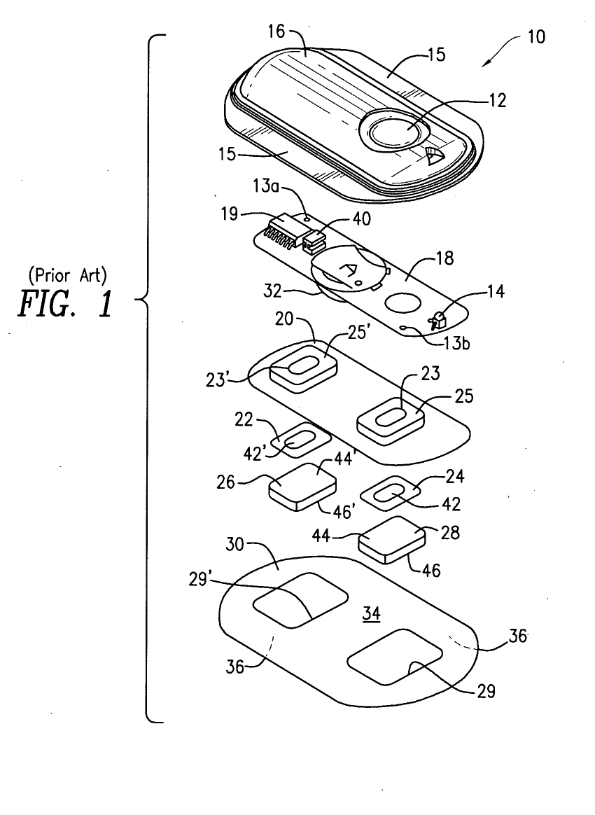

Electrotransport devices, such as iontophoretic devices, are known in the art, e.g., U.S. Pat. No. 6,216,033. The structures, drugs, and electrical features of U.S. Pat. No. 6,216,033 and in FIG. 1 can be adapted to be used in the present invention, as can be understood by one skilled in the art.

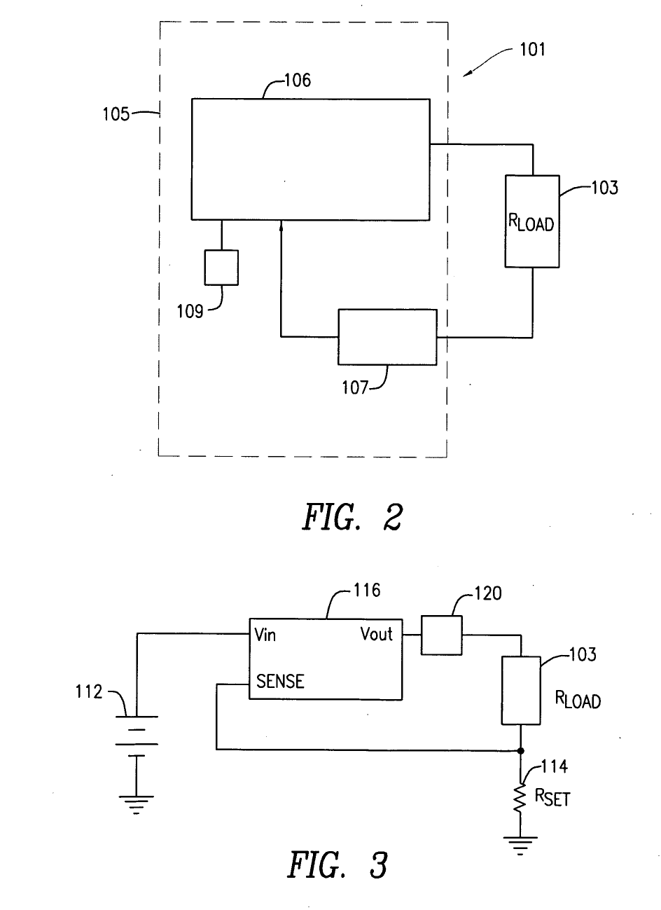

FIG. 2 is a block diagram representing the present invention. In the iontophoretic drug delivery system 101, a load 103, e.g., skin (represented by resistance RLOAD) is connected to a control circuit 105 that includes a power supply such as a battery 109 the voltage of which is boosted by booster circuit 106 to provide the voltage output to the load 103. The control circuit 105 can also include a feedback sensor that senses current and returns a voltage to the booster circuit 106 as feedback to control the current to a constant value for a period. The downstream voltage of the load 103 is fed to the current sensor 107 in the control circuit 105 for feedback control to provide a constant current over a period of time to the load 103.

In an iontophoretic drug delivery system, the resistance from the electronics to the electrodes and the reservoirs is very small compared to the resistance of the skin and can be taken as negligible in calculating the current, voltage, and resistance of the system. However, if desired, such resistance of the electrodes and the reservoirs can be measured and taken into account.

FIG. 3 illustrates a schematic representation of an embodiment of the present invention. In the system shown in FIG. 3, the load 103, e.g., skin (represented by resistance RLOAD) is connected to a booster circuit 116, e.g., commercially available boost converter switching regulator, such as LT3464 (LT3464ETS8, Linear Technology, Milpitas, Calif.), that boosts the voltage of a battery 112 to a higher voltage to supply the load 103 and a set resistor (feedback sensor) 114. It is understood that other switching regulators, e.g., MAX8571 (MAX8571EUT, Maxim Integrated Products, Sunnyvale, Calif.) or TPS61041 (TPS61041DBV, Texas Instruments, Dallas, Tex.), can easily be adapted for this application by one skilled in the art. The downstream voltage of the load is connected to the feedback input of a boost converter switching regulator 116 to provide feedback to the boost converter switching regulator 116 to boost the battery voltage (which is feed to VIN voltage input of the boost converter switching regulator 116) to result in a boosted VOUT output voltage to the load 103. The output current of the boost converter 116 switching regulator is set by the ratio Vsense/Rset. A semiconductor circuit component (preferably a discrete semiconductor component) 120 is connected in series with the VOUT and the load to impose a voltage drop such that the voltage at the VOUT required to drive the constant current will always be larger than the voltage of the power supply, regardless of how much the load resistance falls. For example, the semiconductor circuit component 120 can be a Zener diode in reverse biased position or a MOSFET through which current is passed from the VOUT of the boost converter 16 in series to the load. For simplicity of design and implementation, it is preferred that only one semiconductor circuit component is present at the output of the boost converter, such as only one Zener diode. Because the input impedance of the boost converter is very high at the feedback node, practically all the current that flows through the semiconductor circuit component (e.g., Zener diode) flows through the load and the feedback sensor. Any current from the output of the boost converter not passing through that path is negligible (e.g., for the LT3464, this current is 3 to 30 nA). Thus, the same current is considered to pass through the semiconductor circuit component, through the load (body surface) and the feedback sensor (e.g., set resistor). It is noted that FIG. 3 is schematic and does not show certain components that may be necessary or useful for proper or improved operation of off-the-shelf boost converters, such as a Schottky diode, an inductor, and an output capacitor. These are not included in the diagram for the sake of clarity of the drawing, since such components related to boost converters are well understood by those skilled in the art and can be easily implemented in view of the present disclosure by such skilled practitioners.

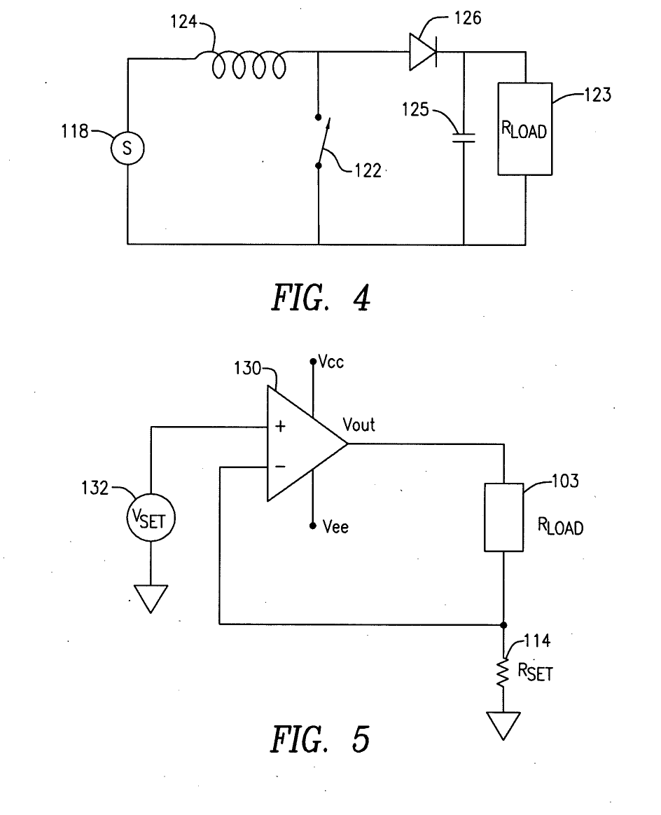

For the booster circuit, a boost converter (step-up converter), preferably commercially available off-the-shelf boost converters, can be used. Boost converters for stepping up voltage are well known to those skilled in the art of circuit design. A boost converter is a power converter with an output dc voltage greater than its input dc voltage. It can take a power supply's voltage at its input and step it up to a higher voltage for its output. It is a class of switching-mode power supply (SMPS). Typically a boost converter contains at least two semiconductor switches (a diode and a transistor) and at least one energy storage element (often containing an inductor and a capacitor). Filters made of inductor and capacitor combinations are often added to a converter's output to improve performance. The switching of the switches allows the current to flow in ways that charge up the voltage of a storage element such as capacitor to provide the stepped up voltage. It is noted that typical booster circuits and devices commonly known in the art can be used to provide boosted voltages and supply constant current. FIG. 4 illustrates the schematic representation of a simple boost converter. In FIG. 4, the power is supplied by a power supply 118 to step up to provide a higher voltage to the load 123. The switching on and off of the switch 122 (typically a transistor switch) controls storage of energy in the inductor 124 and the transfer of its energy to the capacitor 125 through diode 126, which allowing current to flow in only one direction acts in conjunction with the capacitor 125 as a rectifier. In such a boost converter, the output voltage can never fall below that of the input voltage. The booster circuits in prior publications, e.g., U.S. Pat. Nos. 5,804,957, 6,150,802, and 6,842,640, can be adapted to be used in the present invention. Each of these implementations uses the broad concept of a dynamically boosted output voltage varied to maintain constant current through the load. In all three, the discrete semiconductor device, which could be a Zener diode or a PMOS transistor, can be placed in series with the load similarly as shown in FIG. 3.

The presence of the semiconductor circuit component 120 at the VOUT of the booster circuit such as boost converter 116 provides great advantages for iontophoretic drug delivery (see FIG. 3). In a typical constant current supply circuitry (voltage control current source VCCS), e.g., one schematically shown in FIG. 5, connected in a negative feedback configuration, an operational amplifier (op-amp) 130 will try to make VOUT whatever voltage necessary to make the input voltages, i.e., non-inverting input (VSET) from the reference control 132 and inverting input (feed back voltage) from the upstream of feedback sensor (Resistor RSET) 114 as nearly equal as possible. Vcc is the positive power supply and Vee is the negative power supply to the op-amp 130. Since the input impedance of the op-amp 130 is very high, the current through the load is essentially the same that passes through the feedback sensor (resistor) 114 and is therefore held constant. However, in this configuration, the supply voltage Vcc of the op-amp is required to be at least as high as the desired output current multiplied by the sum of the load and set resistors (assuming that the op-amp is capable of rail-to-rail operation at the output). For some op-amps that are not capable of rail-to-rail operation the supply voltage may need to be even higher. For example, if the load resistance is 150 kΩ and the feedback sensor, or set resistance (RSET) is 1 kΩ, and the output current is required to be 200 μA (or 20 mcA), the supply voltage must be at least 30V. For this reason, a booster circuit is needed to boost the supply voltage (typically from a battery with less than 10 volts) to a higher voltage.

For boosting voltage, many implementations of booster circuits as known to those skilled in the art of voltage boosting can be used for an iontophoretic drug delivery device. The application specific integrated circuit (ASIC) based approaches minimize circuit board area by using few external components, but are not optimal in terms of cost (ASICs also generally involve extensive production and prototyping time). Microcontroller based off-the-shelf approaches minimize cost, but use many passive and active discrete components. We have found that one implementation that appears to optimize board area and cost simultaneously is the use of an off-the-shelf boost converter configured as a current source as shown in FIG. 3 discussed above.

The circuit according to FIG. 3 is based on constant-current mode operation of boost converters. It uses a minimal number of off-the-shelf components and, in the case of a switching regulator with external reference control, can be used to supply an adjustable current to varying loads with “smart boosting” of the battery voltage. Many switching regulators have a feature that allows the user to externally control the threshold voltage for the internal comparator at the “sense” or “feedback” pin. Since the ratio of this voltage to the set resistor determines the value of output current, it can be used to adjust the current level. Boost converters are very efficient. Thus, the present electrotransport system is also energy efficient, constrained by the efficiency of the commercial boost converter and generally having an energy efficiency of 65% or above (even for load currents less than 0.5 mA). The power loss in the semiconductor circuit component can be as low as 2 to 5 mW.

The circuit of FIG. 3, however, if without the semiconductor circuit component 120, will not work properly to provide a constant current for electrotransport, such as iontophoretic drug delivery, in which the load resistance can vary greatly. The reason is the fundamental limitation of the boost converter as a controller: the output voltage can never be lowered to less than the battery voltage. In the type of boost converter more commonly available and is preferably used in the present invention, the output voltage is a multiple (which can be digital or fractional, e.g., 8, 5, 2.5, 1.4, etc. but not less than 1) of the input voltage. Using such boost converters without more, if the load resistance falls, the output voltage will fall to a minimum about equal to the input voltage. Such more conventional readily commercially available boost converters have the advantage of being small in size and relatively inexpensive. Such commercially available “off-the-shelf” boost converters include LT3464, MAX8571, and TPS61041 (they are very inexpensive and in the year 2007 A.D. cost about a $1.00 each). Thus, if the load resistor drops low enough such that the output voltage must be lower than the battery voltage to source the desired constant current, the boost converter will actually source a higher current to the load than the desired constant current. For example, if the battery voltage is 3V, the desired current 200 μA, the set resistor is 1 kΩ, and the load resistor is 5 kΩ, the output current will be 500 μA because the output voltage of the boost converter cannot fall below the input voltage (3V in this example). As a result, 2.5 times the desired current is delivered. In iontophoretic delivery, e.g., the delivery of the narcotic fentanyl, a delivery of 250% of the desired dosage of drug could result in severe consequences. Thus, measures need to be implemented to avoid such over-delivery.

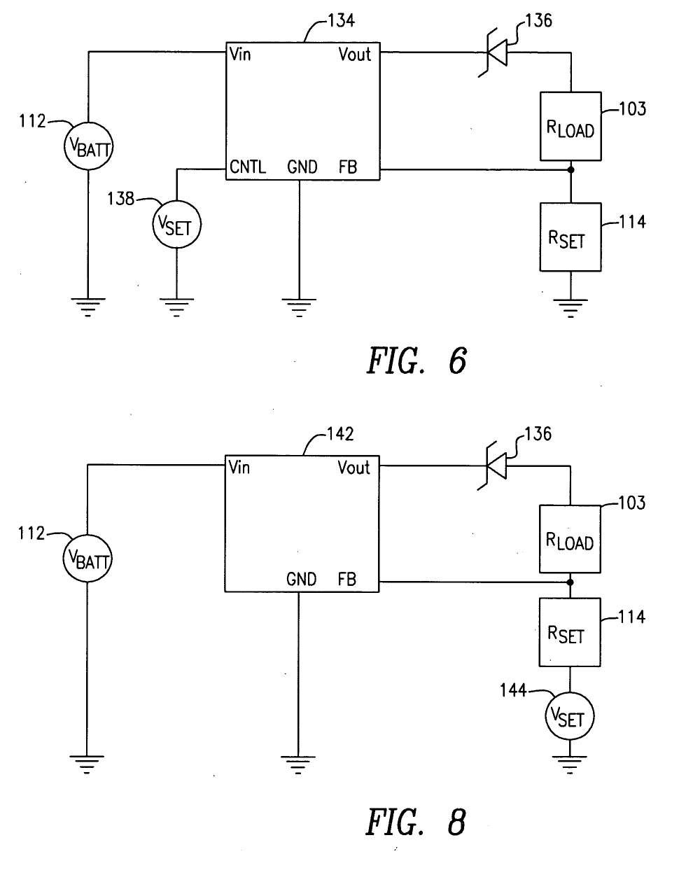

FIG. 6 shows an embodiment of the present invention in which a Zener diode is used as the semiconductor circuit component in a current controller for controlling the current that can pass through the load. A power supply 112, e.g., a battery, supplies power to the boost converter switching regulator 134 to step up to an output voltage VOUT. The output voltage of the boost converter switching regulator is connected through a Zener diode 136 in reverse bias in series to the load 103, e.g., skin (represented by resistance RLOAD). The Zener diode 136 is connected downstream (i.e., current flow direction in normal operation during electrotransport) to a sensor load RSET (e.g. resistor) 114 and also to the Feedback (FB) pin of the boost converter switching regulator 134. An external reference control 138 provides a voltage VSET to set the current to be provided at the output VOUT of the boost converter 134. The current can be adjusted via the external reference control 138 (at VSET) to the switching regulator and can be varied, for example, with the digital to analog converter (DAC) output of a microcontroller. The ability to control the level of the constant current is useful in drug delivery because it may be desirable to deliver different dose of drug during different periods of time. In such a case, after delivery at a particular dose (or current) for a while, the dose (or current) can be adjusted to a different level for another period of time. Furthermore, the adjustment of current levels can be done automatically by programmable electronics such as a microprocessor.

The Zener diode 136 is placed in reverse biased configuration so that it imposes a substantially constant voltage drop between the output of the boost converter 134 and the load 103. The Zener diode, being in series with the load (e.g., skin resistance), clamps the output voltage of the boost converter at a minimum equal to or slightly above the voltage of the power supply (e.g., battery voltage) even in the instance that the load resistance is short circuit. This circuit is capable of sourcing an adjustable constant current of 50 μA to 10 mA to load resistances varying from 500Ω to 650 kΩ with at least 65% efficiency from a 3V power supply (e.g., power source such as a battery). This covers practically all the skin resistance variation in human skin to be treated by iontophoretic drug delivery. Such systems can be implemented at low cost (for less than $2.00) in large quantities, and are so small that a constant current system can be placed on a board area of approximately 1 square centimeter. A Zener diode when connected in this reverse bias way provides a stable voltage drop thereacross. The RSET connected in series with the Zener diode is provided that the Zener diode is in reverse breakdown to provide a stable voltage drop over the range of variation of the resistance of the load. The selection of the Zener diode and RSET is within the skill of those skilled in the art.

As an illustration, in an embodiment, the power supply 12 VBATT, say a battery, provides 3 volts to the boost converter switch regulator. The Zener diode is selected to provide in reverse bias break down voltage at Vz (3V) such that the voltage difference between VOUT and VFB (i.e., the sum of Vz+Ii*RLOAD wherein Ii is the current through the Zener diode and the load RLOAD). Thus the difference between VOUT and VFB is always (3V+Ii*RLOAD), which is always larger than the VIN. Therefore, even if the load resistance falls to zero, the current required to pass through the load will always result in a VOUT from the boost converter switch regulator higher than the voltage of the power supply. If 1 mA current is required to pass through the load, if the RSET is 1 kΩ, when the load resistance is zero, the VOUT will be (3V+1 mA*1 kΩ), i.e., 4V. If the load is 650 kΩ, the VOUT will be (3V+1 mA*(20 kΩ+1 kΩ))=24V. Typically, for iontophoretic drug delivery, depending on the drug, a current between 10 μA to 1 mA may be used. In that case, the power loss though the Zener diode is Ii*Vz=3 mW. Typically, the electrotransport system includes a limiting circuitry to stop drug delivery or display an alarm when the load resistance is above a predetermined threshold value, e.g., such that a higher than desired voltage is required to drive the constant current, e.g., when the VOUT needs to be above 50V.

Generally, the device is designed for use in the normal working range of the load resistance as being 500Ω (generally the skin resistance does not drop below 1 kOhm) to 650 kΩ (e.g., the upper end of skin resistance in iontophoretic delivery about this range). During normal operation, with the skin resistance being in this range, a constant current is delivered to the skin to deliver the desired drug dose. If the load resistance falls, the Zener diode still imposes an about constant voltage drop and therefore acts with a constant resistance when a constant current is delivered. A main achievement in the present invention is the capability to cover such a wide range of currents and load resistances efficiently with minimal error. If the resistance falls below the normal working range, the device with the Zener diode is still able to provide the current of the desired magnitude because the Zener diode imposes a voltage drop on the output of the booster circuit so the boosted voltage never falls below the input voltage of the booster circuit. In the event that the skin resistance goes above the range, it may reach a point at which the booster circuit will no longer be able to supply adequate voltage to drive a constant current. At that time, the device is no longer able to maintain a constant current and the current output to the load will fall. The device can be designed to display an alarm, either by sound or light or both, and stop current delivery when the skin resistance is too large

Generally, the semiconductor circuit component and the feedback sensor are selected such that the sum of the voltage drop across the feedback sensor, the load, and the semiconductor circuit component (such as the Zener diode) at the constant current are always equal or larger than the voltage of the power supply voltage input to the boost converter, regardless of the resistance change of the skin, considering that skin resistance is practically never zero. However, to preclude accidents that can happen in case the skin resistance falls to an extremely low value, preferably, the semiconductor circuit component and the feedback sensor are selected such that the sum of the voltage drop across the feedback sensor and the semiconductor circuit component (such as the Zener diode), not counting the resistance of the load, are always equal or larger than the voltage of the power supply voltage input to the boost converter, regardless of the resistance change of the skin (even when the skin resistance is zero). Given the voltage of the power supply, the range of working resistance of the skin (e.g., 500Ω to 650 kΩ), and the range of current desired (e.g., 50 μA to 10 mA), a person skilled in the art will be able to readily select the semiconductor circuit component and the feedback sensor.

FIG. 7 illustrates an embodiment of FIG. 6 in further detail. In the electrotransport system of FIG. 7, the boost converter switch regulator is LT3464, the Zener diode imposes a voltage drop of 5.1V, and includes the following pins: GND, OUT, FB, CNTRL (CNTRL is sometimes called CNTL, as they are used interchangeably in literature/commercial applications), CAP, SW, IN, and SHDN. The GND pin is connected to ground. The OUT pin is connected to through the Zener diode to the load RL and the feedback sensor. The FB pin is connected to the downstream of the load RL where load RL is connected to the feedback sensor to provide a feedback voltage to the boost converter switch regulator. The IN pin receives input voltage from the power supply (3V battery) for boosting to a higher voltage for output at the OUT pin. An inductor connected from the power supply to the SW pin provides an energy storage element for transferring energy to boost voltage. A capacitor is connected to the OUT pin for storing energy transferred from the inductor. The CAP pin is specific to this particular boost regulator and allows for disconnecting the load if the output voltage exceeds a threshold value. In most designs, this capacitor may not be necessary. An input from the controller, e.g., microprocessor digital-to-analog-converter (MCU DAC) to the CNTRL pin provides a setting voltage to set the target output constant current at the OUT pin. An input from the microprocessor unit (MCU) in the electrotransport device to the SHDN pin provides instruction to shut down the booster activity of the boost converter switch regulator. Such a system can be used for iontophoretic drug delivery with a constant current of 50 μA to 1 mA. Such a system will be able to deliver a constant current for delivery for skin resistance at about 500Ω to 650 kΩ.

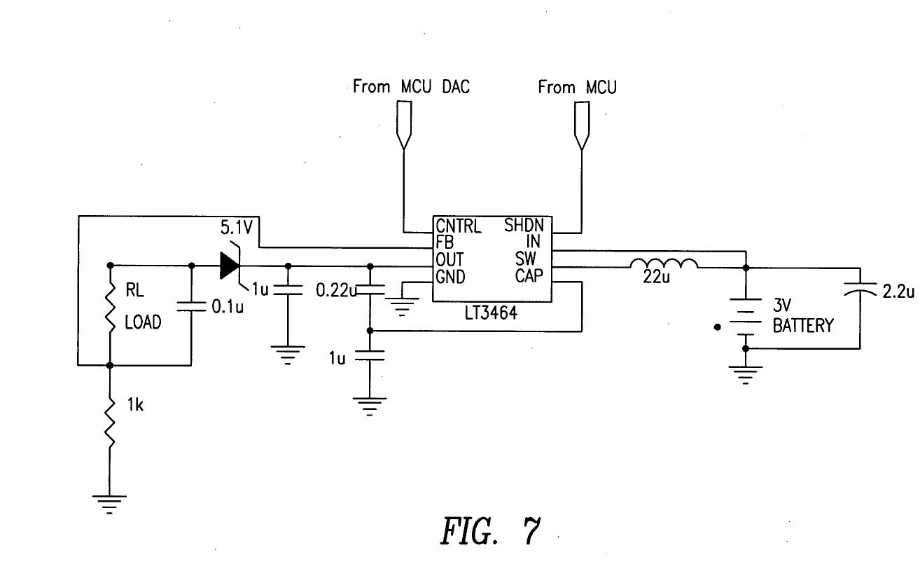

FIG. 8 shows another embodiment in which the level or magnitude of the constant current controlling reference voltage is not provided to a pin in the boost converter switch regulator, such as when the boost converter switch regulator lacks such a CNTRL pin. In this case, no controlling reference voltage is connected to any CNTRL pin in the boost converter switch regulator. Rather, a controlling reference voltage supply (VSET) 144 is connected to lift (or modulate) the voltage of the sensor resistance 114 from ground an additional amount. Since the feedback voltage is internally fixed to a set potential above ground (usually 1.2V), the voltage across the sense resistor will be fixed at the internal reference voltage less the set voltage. Accordingly, the current through the resistor can be dynamically varied by this set voltage. In this implementation, current will increase as set voltage decreases.

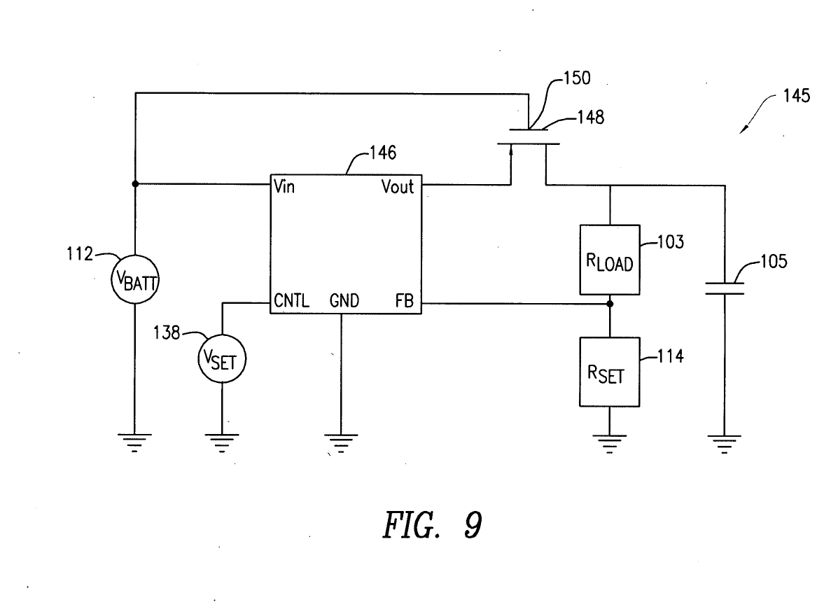

FIG. 9 shows a schematic representation of another embodiment of the control current delivery system of the present invention. In FIG. 9, the iontophoretic drug delivery system 145 includes a boost converter switch regulator 146 with a PMOS FET (P-type Metal Oxide Semiconductor Field Effect Transistor) 148 connected to the VOUT pin of the boost converter switch regulator to pass current therefrom to the load (RLOAD) 103. (Of course, it is to be understood that nowadays, MOS devices are not necessarily made with metal and oxide, but many in fact have polycrystalline silicon for the gate.) The power supply battery 112, the feedback sensor 114 and the external reference control 138 are connected to the boost converter switch regulator 146 in a manner similar to FIG. 6. The gate 150 of the PMOS FET is connected to the VIN pin of the boost converter and the power supply. The source is connected to the VOUT pin and the drain is connected to the load 103. The capacitor 105 connected parallel to the load 103 and feedback resistor 114 to ground is optionally present to help to smooth the output voltage to reduce ripple. During normal operation of the system 145, the PMOS FET is highly conductive. In a PMOS FET with P-Channel, when an adequately negative gate-source voltage (compared with positive source-gate voltage) is applied, current is conducted when the gate potential is low enough to repel electrons from the channel into the bulk, leaving behind a conductive interface between the source and the drain regions comprised of p-type carriers, holes. When a near-zero or positive voltage is applied between the gate and the body of the semiconductor device PMOS FET, no current can flow between the source and the drain. The PMOS FET increases its resistance and therefore the source-to-drain voltage drops when the output voltage of the boost converter switch regulator 146 (thus the input to the source of the PMOS FET) approaches the battery voltage VBATT (the same as the voltage at the gate), which happens when the RLOAD is very small. If the output voltage VOUT approaches the battery voltage, then the resistance of the PMOS FET increases until an equilibrium point is reached. This point is when the output voltage is at a threshold voltage higher than the battery voltage. When the load resistance becomes small, the PMOS FET imposes a resistance and an increasing voltage drop from the output voltage of the booster circuit. For example, if the output current is set to 500 μA, the PMOS drain to source resistance (for a typical device) will be 6.9 kΩ, 6 kΩ, and 20 mΩ respectively for corresponding load resistances of 100Ω, 1 kΩ, and 10 kΩ. With the PMOS FET present, the output voltage of the booster circuit never falls below the input voltage, no matter how small the load resistance is. Thus, the system is able to deliver a constant current to the load even if the load resistance changes substantially, e.g., between 500Ω to 650 kΩ, for, e.g., 50 micro-amps of current flow.

It is noted that the setting of the reference control voltage to control the magnitude of the constant current during a period of drug delivery using the PMOS FET as the semiconductor circuit component can be done with a VSET connected to the CNTRL pin of the switch regulator or by lifting the voltage of the sensor resistance RSET as was described above in FIG. 6 and FIG. 8 for the embodiments having the Zener diode as the semiconductor circuit component. Furthermore, it is also noted that the load RLOAD does not necessarily have to be downstream in terms of current flow from the semiconductor circuit component. The semiconductor circuit component can also be placed downstream of the load. In the case of using a PMOS FET, one would provide a lower voltage than the battery voltage (e.g., by dividing the battery voltage) to the gate of the PMOS FET so that the gate voltage controls the switching of the PMOS FET to provide a higher resistance by the PMOS FET when the load resistance is small.

As mentioned above, the present systems with the semiconductor circuit component for imposing a voltage drop is very energy efficient, greater than 60% and typically between 65% and 75%, and efficiency is mostly determined by the switching regulator itself since no other integrated circuits are used. The regulators mentioned above (LT3464, MAX8571, and TPS61041) are very efficient, even down to lower currents, usually above 75% for the current ranges of interest. Although the Zener diode will provide a small loss in efficiency, such a loss is much less than any other implementation using op-amps or other integrated circuits. The PMOS approach is even more efficient, since the transistor will only drop a voltage across it in the case that the load resistance falls too low. This is the most efficient addition to the simple switching regulator which also provides for lower load resistances. For simplicity of design and implementation and for energy efficiency, it is preferred that only one semiconductor circuit component (such as only one PMOS FET or Zener diode) is present at the output of the boost converter connecting to the load.

In view of the present disclosure, one skilled in the art will know that the use of a semiconductor circuit component such as a Zener diode or a PMOS FET for imposing a voltage drop to allow the device to continue delivery of constant current can be adapted into prior systems with booster circuits not already described in the figures of the present disclosure. For example, in WO 99/30773, FIG. 9, the comparator, analog switch, and resistor can be replaced by a single Zener diode or a PMOS FET which can dynamically respond to low skin resistances. In U.S. Pat. No. 6,150,802, a semiconductor circuit component can be placed in series with the load resistance to ensure that the current will still be controlled if the load resistance decreases.

A suitable electrotransport device can include typical features of an electrotransport system such as electrodes, drug reservoirs, and the like. For example, the system can contain an anodic donor electrode, e.g., one that contains silver, and a cathodic counter electrode, e.g., one that contains silver chloride. The donor electrode is in electrical contact with the donor reservoir containing the aqueous solution of a drug salt, e.g., fentanyl salt. The donor reservoir is preferably a hydrogel formulation. The counter reservoir also preferably contains a hydrogel formulation containing a (e.g., aqueous) solution of a biocompatible electrolyte, such as citrate buffered saline.

The reservoirs of the electrotransport delivery devices generally can contain a gel matrix, with the drug solution uniformly dispersed in at least one of the reservoirs. In an IONSYS system, the gel was made from poly (vinyl alcohol). Obviously, other types of reservoirs such as membrane-confined reservoirs are possible and contemplated. The application of the present invention is not limited by the type of reservoirs used. Gel reservoirs are described, e.g., in U.S. Pat. Nos. 6,039,977 and 6,181,963, which are incorporated by reference herein in their entireties. Suitable polymers for the gel matrix can contain essentially any synthetic and/or naturally occurring polymeric materials suitable for making gels. A polar nature is preferred when the active agent is polar and/or capable of ionization, so as to enhance agent solubility. Optionally, the gel matrix can be a water swellable nonionic material.

Examples of suitable synthetic polymers include, but are not limited to, poly (acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

The reservoir formulation for transdermally delivering cationic drugs by electrotransport is preferably composed of an aqueous solution of a water-soluble salt, such as HCl or citrate salts of a cationic drug, such as fentanyl. More preferably, the aqueous solution is contained within a hydrophilic polymer matrix such as a hydrogel matrix. The drug salt is preferably present in an amount sufficient to deliver an effective dose by electrotransport over a delivery period to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl with system having fentanyl salt (e.g., hydrochloride salt) has been described in U.S. Pat. No. 6,171,294, which is incorporated by reference herein. The parameters such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

A preferred hydrophilic polymer matrix is polyvinyl alcohol such as a washed and fully hydrolyzed polyvinyl alcohol (PVOH), e.g. MOWIOL 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRLIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments, can be about 10% to about 30%, preferably about 15% to about 25%, and more preferably about 19%. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90° C. to 95° C. for at least about 0.5 hour. The hot mix is then poured into foam molds and stored at freezing temperature of about −35° C. overnight to cross-link the PVOH. Upon warming to ambient temperature, a tough elastomeric gel is obtained suitable for ionic drug electrotransport.

A variety of drugs can also be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

Some ionic drugs are polypeptides, proteins, hormones, or derivatives, analogs, mimics thereof. For example, insulin or mimics are ionic drugs that can be driven by electrical force in electrotransport.

For more effective delivery by electrotransport, salts of such analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art. Likewise, system for delivery anionic drugs with cationic counter ions can be made.

A device according to the present invention can be made by forming the various parts of the device (e.g., the parts as shown in FIG. 1) and assembling the parts into an assembled device. The polymeric layers such as the housing parts can be made by molding. Some of the layers can be applied together and secured. Some of the parts can be affixed together by adhesive bonding or mechanical anchoring. Such chemical adhesive bonding methods and mechanical anchoring methods are known in the art. A device that delivers fentanyl with a more stable flux can be made as a single unit as shown in FIG. 1. Such a device is made by assembling the parts together to form a unit and then the assembled unit is packaged and stored until it is to be removed from the packaging for use. Alternatively, a device can be made by first making an electronic module and a reservoir module separately, wherein the two modules can be stored separately (e.g., in protected packages). The electronic module contains the control electronics and the reservoir module contains the reservoirs. The electronic module can be coupled with the reservoir module just before use by a medical professional, e.g., by inserting one module into the other module or by pressing the modules together. For example, the electronic module can be reusable whereas the reservoir module is disposable. After the system has been used, the reusable electronic module can be separated from the reservoir module and reused again by coupling with a fresh reservoir module. The used reservoir module can be discarded according to proper standard procedure. Iontophoretic drug delivery systems each with a separate drug reservoir unit and controller unit have been described in the past in patent and scientific literature. Such prior separate systems can be adapted for the electronic modules and reservoir modules of the present invention. For example, such assemble-before-use systems (electrotransport devices having parts being connected together before use) include those described in, U.S. Pat. No. 5,320,597 (Sage, Jr. et al); U.S. Pat. No. 4,731,926 (Sibalis); U.S. Pat. No. 5,358,483 (Sibalis); U.S. Pat. No. 5,135,479 (Sibalis et al.); U.S. Pat. No. 5,919,155 (Lattin et al.), U.S. Pat. No. 5,445,609 (Lattin et al.); U.S. Pat. No. 5,603,693 (Frenkel et al.); UK Patent Publication GB2239803 (Devane et al); and WO1996036394 (Lattin et al.).

The electronics including circuit broad can be fabricated with common circuit broad manufacturing techniques that are well known in the art. The boost converters can be off-the-shelf units available from semiconductor device manufacturers, as are the semiconductor circuit components such as the Zener diode and PMOS FET. The printed circuit board with additional electrical components, if any, can be connected with electrodes, reservoirs, etc and placed in housing parts to provide a device similar to that shown in FIG. 1, or other similar electrotransport devices. Knowing the design of the voltage boosting to provide a constant current disclosed herein, a person skilled in the art will be able to adapt the design to make electrotransport devices.

In the use of electrotransport drug delivery, e.g., iontophoretic delivery systems similar to the show in FIG. 1, a constant current can be applied to deliver drug for a period of time to achieve certain dose. For example, the IONSYS system (which contains fentanyl hydrochloride) can be used to deliver fentanyl ions. Typically a constant current is conducted for a dose period of 10 minutes to deliver a dose in IONSYS system. An electrotransport system can be made to deliver a constant current for a period of time. The period can be made to be different if desired. Systems can also be implemented in which the dosage may differ from one period to another. During each period of constant current delivery, the present current control schemes can be used which can include one or more dose periods.

In another example of alternative design, a circuit similar to that shown in FIG. 7 can be made by using a digitally controlled potentiometer in place of the sense resistor. This potentiometer's resistance can be dynamically adjusted to select the appropriate current. This embodiment can be used for boost converters without an external reference control pin. The above-described exemplary embodiments are intended to be illustrative in all respects, rather than restrictive, of the present invention. Thus the present invention is capable of many variations in detailed implementation that can be derived from the description contained herein by a person skilled in the art, e.g., by permutation or combination of various features. Although specific iontophoretic devices are described in detail as illustration, other modifications are possible by one skilled in the art. All such variations and modifications are considered to be within the scope of the present invention. The entire disclosure of each patent, patent application, and publication cited or described in this document is hereby incorporated herein by reference.

What is claimed is:

1. A transdermal electrotransport system for administering a drug through a body surface of a user, comprising:
(a) donor reservoir comprising an electrotransportable drug;
(b) a first electrode and a second electrode for conducting a current to flow from the first electrode to the second electrode through the donor reservoir and the body surface to drive the electrotransportable drug from the donor reservoir transdermally by electrotransport; and
(c) a controller for controlling the current, the controller connected to the first electrode and the second electrode to provide the current for electrotransport, the controller containing a booster circuit capable of boosting the voltage of a power supply to a higher voltage, a feedback sensor, and a semiconductor circuit component electrically connected with the booster circuit and the first and second electrodes so the same current flows through the semiconductor circuit component, the feedback sensor and the body surface, the feedback sensor providing a feedback voltage to the booster circuit for feedback control to provide a constant current during a delivery period while accommodating changes in resistance through the body surface, the semiconductor circuit component maintaining the sum of voltage across the semiconductor circuit component, the body surface, and the feedback sensor to be always at least equal to the voltage of the power supply.

2. The system of claim 1, wherein the semiconductor circuit component is selected from the group consisting of a field effect transistor (FET) and a Zener diode, and wherein the sum of voltage across the semiconductor circuit component, the body surface, and the feedback sensor is at least equal to the voltage of the power supply even if the sum of voltage across the body surface and the feedback sensor fall below the voltage of the power supply.

3. The system of claim 2, wherein the semiconductor circuit component is either a PMOS FET or a Zener diode and the booster circuit includes a boost converter with semiconductor switch, the boost converter boosting an input voltage to always be larger than the input voltage during operation to result in an output voltage.

4 . The system of claim 3 wherein the sum of voltage across the semiconductor circuit component and the feedback sensor is at least equal to the voltage of the power supply even if the sum of voltage across the body surface and the feedback sensor falls below the voltage of the power supply.

5. The system of claim 3 wherein the semiconductor circuit component is a Zener diode having only one cathode and only one anode in reverse bias.

6. The system of claim 3, wherein the semiconductor circuit component is a PMOS FET and has only one gate, one source and one drain, wherein the gate is at a higher voltage than the source and the gate is at a voltage equal to the voltage of the power source, and wherein the voltage of the source is always no less than the voltage of the power supply during operation.

7. The system of claim 2, wherein the semiconductor circuit component is positioned so that current flows from the semiconductor circuit component to the body surface and the feedback sensor.

8. The system of claim 2, wherein the controller controls the current delivery to never permit a current higher than a predetermined current to pass through the body surface.

9. The system of claim 3, wherein the controller provides the constant current while tolerating the body surface to vary in resistance from 500 ohm to 650 kohm.

10. The system of claim 3, wherein the controller includes a switching regulator having an in pin for receiving a voltage from the power supply, a feedback pin to receive feedback control voltage from the feedback sensor, an out pin to provide a constant current out to the body surface, and a control pin to receive a reference voltage to set the current to a constant value to the body surface as long as the body surface has a resistance from 500 ohm to 650 kohm.

11. The system of claim 3, wherein the controller includes a switching regulator having an in pin for receiving a voltage from the power supply, a feedback pin to receive feedback control voltage from the feedback sensor, and an out pin to provide a constant current out to the body surface, wherein a reference voltage is provided to the feedback sensor to control the current to a constant value to the body surface as long as the body surface has a resistance from 500 ohm to 650 kohm.

12. The system of claim 3, wherein power loss in the semiconductor circuit component is between 2 to 5 mW.

13. The system of claim 3, wherein power loss in the semiconductor circuit component increases with decreasing body surface resistance.

14. The system of claim 3, wherein the controller controls the current delivery in discrete periods of constant current delivery at different levels of current.

15. A method for controlling current in a transdermal electrotransport device for delivery of an electrotransportable drug through the body surface, comprising:
controlling current delivery to a drug reservoir to drive ions of an electrotransportable drug therefrom by boosting an input voltage with a booster circuit to a higher voltage output voltage for driving a current through the body surface, wherein a semiconductor circuit component and a feedback sensor are connected electrically with the body surface and the booster circuit so that the same current flows through the semiconductor circuit component, the feedback sensor and the body surface, the feedback sensor providing a feedback voltage to the booster circuit for feedback control to provide a constant current while accommodating body surface resistance change, and the semiconductor circuit component imposing a voltage drop to maintain the sum of voltage across the semiconductor circuit component, the body surface, and the feedback sensor to be always at least equal to the voltage of the power supply regardless of the resistance change in the body surface.

16. The method of claim 15, wherein the semiconductor circuit component is selected from the group consisting of a field effect transistor (FET) and a Zener diode, and wherein with the constant current the sum of voltage across the semiconductor circuit component, the body surface, and the feedback sensor is at least equal to the voltage of the power supply even if the sum of voltage across the body surface and the feedback sensor fall below the voltage of the power supply during operation of the device.

17. The method of claim 16, including selecting either a PMOS FET or a Zener diode as the semiconductor circuit component and wherein the booster circuit has a boost converter with semiconductor switch, the boost converter boosting an input voltage to always be larger than the input voltage to result in an output voltage for driving electrotransport.

18. The method of claim 16, wherein with the constant current the sum of voltage across the semiconductor circuit component and the feedback sensor is at least equal to the voltage of the power supply even if the sum of voltage across the body surface and the feedback sensor falls below the voltage of the power supply.

19. The method of claim 17, wherein the semiconductor circuit component is a Zener diode and has only one cathode and only one anode in reverse bias.

20. The method of claim 17, wherein the semiconductor circuit component is a PMOS FET having only one gate, one source and one drain wherein the gate is at a higher voltage than the source and the gate is at a voltage equal to the voltage of the power source, and wherein the voltage of the source is always no less than the voltage of the power supply during electrotransport.

21. The method of claim 16, including positioning the semiconductor circuit component so that current flows from the semiconductor circuit component to the body surface and the feedback sensor.

22. The method of claim 16, wherein the controller controls the current delivery to never permit a current higher than a predetermined current to pass through the body surface.

23. The method of claim 17, wherein the controller provides the constant current while accommodating the body surface to vary in resistance from 500 ohm to 650 kohm.

24. The method of claim 17, wherein the controller includes a switching regulator having an in pin for receiving a voltage from the power supply, a feedback pin to receive feedback control voltage from the feedback sensor, an out pin to provide a constant current out to the body surface, and a control pin to receive a reference voltage to set the current to a constant value to the body surface as long as the body surface has a resistance from 500 ohm to 650 kohm.

25. The method of claim 17 wherein the controller includes a switching regulator having an in pin for receiving a voltage from the power supply, a feedback pin to receive feedback control voltage from the feedback sensor, and an out pin to provide a constant current out to the body surface, and wherein a reference voltage is provided to the feedback sensor to control the current to a constant value to the body surface as long as the body surface has a resistance from 500 ohm to 650 kohm.

26. The method of claim 17, wherein power loss in the semiconductor circuit component is between 2 to 5 mW.

27. The method of claim 17, wherein power loss in the semiconductor circuit component increases with decreasing body surface resistance.

28. The method of claim 17, wherein the controller controls the current delivery in discrete periods of constant current delivery at different levels of current.
